(19) United States
(12) Patent Application Publication
(10) Pub. No.: US 2019/0134606 A1
(43) Pub. Date: May 9, 2019

(54) METHOD FOR PRODUCING AFFINITY SEPARATION MATRIX, AND AFFINITY SEPARATION MATRIX

(71) Applicant: KANEKA CORPORATION, Osaka (JP)

(72) Inventors: Dai Murata, Hyogo (JP); Shinichi Yoshida, Hyogo (JP)

(73) Assignee: KANEKA CORPORATION, Osaka (JP)

(21) Appl. No.: 16/183,878

(22) Filed: Nov. 8, 2018

Related U.S. Application Data
(63) Continuation of application No. PCT/JP2017/016819, filed on Apr. 27, 2017.

(30) Foreign Application Priority Data
May 11, 2016 (JP) 2016-095264

(51) Int. Cl. B01J 20/32; C07K 1/22; C07K 17/02; B01J 20/24; B01J 20/282

(52) U.S. Cl. CPC B01J 20/3274; C07K 1/22; C07K 17/02; B01J 2220/52; B01J 20/282; B01J 2220/80; B01J 20/24

(57) ABSTRACT

A method for producing an affinity separation matrix includes immobilizing a κ chain variable region-binding peptide on a water-insoluble carrier through a terminal cysteine residue of the κ chain variable region-binding peptide. The cysteine residue is located at an N-terminal or a C-terminal of the κ chain variable region-binding peptide. The κ chain variable region-binding peptide is a ligand having an affinity for a κ chain variable region. The affinity separation matrix includes the ligand and the water-insoluble carrier.

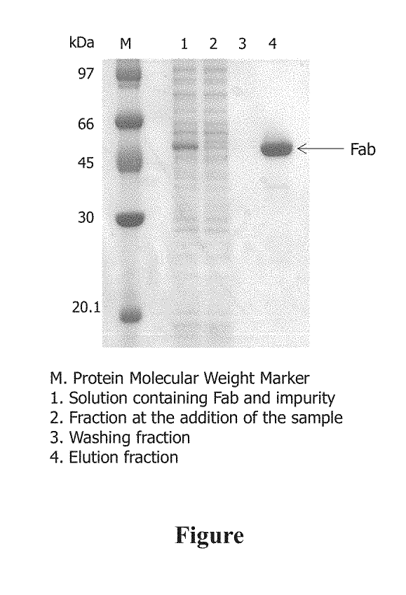

### TECHNICAL FIELD

One or more embodiments of the present invention relate to an affinity separation matrix having a very excellent binding ability to a peptide containing a κ chain variable region, a method for producing the affinity separation matrix, and a method for producing a κ chain variable region-containing peptide by using the affinity separation matrix.

### BACKGROUND

As one of important functions of a protein, an ability to specifically bind to a specific molecule is exemplified. The function plays an important, role in an immunoreaction and a signal transduction in a living body. A technology utilizing the function for purifying a useful substance has been actively developed. As one example of proteins which are actually utilized industrially, for example, Protein A affinity separation matrix has been used for capturing an antibody drug to be purified with high purity at one time from a culture of an animal cell (Non-patent documents 1 and 2). Hereinafter, Protein A is abbreviated as “SpA” in some cases.

An antibody drug which has been developed is mainly a monoclonal antibody, and a monoclonal antibody has been produced on a large scale by using recombinant cell cultivation technology. A “monoclonal antibody” means an antibody obtained from a clone derived from a single antibody-producing cell. Most of antibody drugs which are presently launched are classified into an immunoglobulin G (Ig) subclass in terms of a molecular structure. In addition, an antibody drug consisting of an antibody derivative such as an antibody fragment has been actively subjected to clinical development. An antibody fragment has a molecular structure obtained by fragmenting an immunoglobulin, and various antibody fragment drugs have been clinically developed (Non-patent Document 3).

In an initial purification step of an antibody drug production process, the above-described SpA affinity separation matrix is utilized. SpA is, however, basically a protein which specifically binds to an Fc region of IgG. Thus, SpA affinity separation matrix cannot capture an antibody fragment which does not contain an Fc region. Accordingly, an affinity separation matrix capable of capturing an antibody fragment which does not contain an Fc region of IgG is highly required industrially in terms of a platform development of a process for purifying an antibody drug.

A plurality of peptides which bind to a region except for an Fc region of IgG have been already known (Non-patent Document 4). Among such peptides, a peptide which can bind to a variable region as an antigen-binding domain may be most preferred in terms of many kinds of antibody fragment format to be bound and an ability to also bind to IgM and IgA. As such a peptide, for example, Protein L has been well-known. Hereinafter, Protein L is abbreviated as “PpL” in some cases. PpL is a protein which contains a plurality of κ-chain variable region-binding domains, and amino acid sequences of each κ-chain variable region-binding domain are different from each other. Hereinafter, a κ-chain variable region is abbreviated as “VL-κ” in some cases. In addition, the number of VL-κ-binding domains and the amino acid sequences of each VL-κ-binding domain are different depending on the kind of a strain. For example, the number of VL-κ-binding domains in PpL of Peptostreptococcus magnus 312 strain is 5, and the number of VL-κ-binding domains in PpL of Peptostreptococcus magnus 3316 strain is 4 (Non-patent documents 5 to 7, and Patent documents 1 and 2). There are no domains that have the same amino acid sequence as each other in the totally 9 VL-κ-binding domains.

An affinity separation matrix having PpL as a ligand has been commercially available. In the case of an affinity separation matrix having SpA, which mainly binds to an Fc region of IgG, a technology to control an immobilization of SpA on a carrier by introducing a mutation into SpA has been developed and a technology to improve a binding capacity of a carrier to an antibody has been advanced (Patent documents 3 and 4). On the one hand, in the case of an affinity separation matrix having PpL, which binds to VL-κ, an attempt to control an immobilization of PpL on a carrier by introducing a mutation into PpL is not sufficient and there remains room for improvement on a binding capacity of an affinity separation matrix having PpL as a ligand to a target.

### PATENT DOCUMENT

Patent Document 1: JP H7-506573 T

Patent Document 2: JP H7-507682 T

Patent Document 3: WO 1997/017361

Patent Document 4: JP 2007-252368 A

### NON-PATENT DOCUMENT

Non-patent Document 1: Hober S., et al., J. Chromatogr. B, 2007, vol. 848, pp. 40-47

Non-patent Document 2: Shukla A. A., et al., Trends Biotechnol., 2010, vol. 28, pp. 253-261

Non-patent Document 3: Nelson A. N., et al., Nat. Biotechnol., 2009, vol. 27, pp. 331-337

Non-patent Document 4: Bouvet P. J., Int. J. Immunopharmac., 1994, vol. 16, pp. 419-424

Non-patent Document 5: Kastern W., et al., J. Biol. Chem., 1992, vol. 267, pp. 12820-12825

Non-patent Document 6: Murphy J. P., et al., Mol. Microbiol., 1994, vol. 12, pp. 911-920

Non-patent Document 7: Housden N. G., et al., Biochemical Society Transactions, 2003, vol. 31, pp. 716-718

As described above, SpA affinity separation matrix to adsorb an antibody to be purified has been conventionally put to practical use and a product having a high binding capacity to an antibody has been developed due to an advance of genetic engineering technology and protein engineering technology. On the one hand, an antibody fragment which does not contain an Fc region has been actively studied and developed in recent years but cannot be purified by SpA affinity separation matrix. Accordingly, an affinity separation matrix on which a ligand having an affinity for an antibody fragment is required. An affinity separation matrix which is commercially available presently and which is used for purifying an antibody containing a κ chain variable region exhibits a lower binding capacity than SpA affinity separation matrix and is required to further improve its performance.

One or more embodiments of the present invention provide an affinity separation matrix having an excellent binding capacity and binding efficiency to a κ chain variable region-containing peptide, a method for producing the affinity separation matrix, and a method for producing a κ chain variable region-containing peptide by using the affinity separation matrix.

### SUMMARY

The inventors intensively studied and completed one or more embodiments of the present invention by finding that an affinity separation matrix prepared by immobilizing a κ chain variable region-binding peptide having cysteine at the end as a ligand on a water-insoluble carrier has very excellent binding capacity and binding efficiency to a κ chain variable region-containing peptide.

Hereinafter, one or more embodiments of the present invention are described.

[1] A method for producing an affinity separation matrix,

wherein the affinity separation matrix contains a ligand having an affinity for a κ chain variable region and a water-insoluble carrier,

comprising the step of immobilizing a κ chain variable region-binding peptide having a cysteine residue at at least one of an N-terminal and a C-terminal as the ligand on the water-insoluble carrier through the terminal cysteine residue.

[2] The method according to the above [1], wherein an amino acid sequence of the κ chain variable region-binding peptide comprises an amino acid sequence of the following formula (I):

(X¹)ₘ—(Y¹)ₚ-Glu-X²-Val-Thr-Ile-Lys-X³-Asn-X⁴—X⁵—X⁶—X⁷—X⁸-Gly-X⁹—X¹⁰-Gln-X¹¹-Ala-X¹²-Phe-Lys-Gly-Thr-Phe-X¹³—X¹⁴-Ala-X¹⁵—X¹⁶—X¹⁷-Ala-Tyr-X¹⁸-Tyr-Ala-X¹⁹—X²⁰-Leu-X²¹-Lys-X²²—X²³-Gly-X²⁴-Tyr-Thr-X²⁵-Asp-X²⁶—X²⁷-Asp-X²⁸-Gly-X²⁹-Thr-X³⁰-Asn-Ile-X³¹-Phe-Ala-Gly-(Y²)_{q}—(X³²)ₙ (I)

wherein X¹ is Cys, X² is Gln or Glu, X³ is Glu, Ala or Val, X⁴ is Ile or Leu, X⁵ is Tyr or Ile, X⁶ is Phe or Tyr, X⁷ is Ala or Glu, X⁸ is Asp ox Asn, X⁹ is Thr, Ser or Lys, X¹⁰ is Val, Ile or Thr, X¹¹ is His, Thr or Asn, X¹² is Thr or Glu, X¹³ is Ala or Glu, X¹⁴ is Glu or Lys, X¹⁵ is Thr or Val, X¹⁶ is Ala or Ser, X¹⁷ is Glu, Asp or Lys, X¹⁸ is Arg or Ala, X¹⁹ is Asp or Asn, X²⁰ is Leu, Thr or Ala, X²¹ is Ser, Lys or Ala, X²² is Glu, Asp or Val, X²³ is His or Asn, X²⁴ is Lys or Glu, X²⁵ is Ala or Val, X²⁶ is Leu or Val, X²⁷ is Glu or Ala, X²⁸ is Gly or Lys, X²⁹ is Tyr, Leu or Asn, X³⁰ is Ile or Leu, X³¹ is Arg or Lys, X³² is Cys, Y¹ and Y² are independently peptide residues having 1 or more and 15 or less amino acid residues, m, n, p and q are independently 0 or 1, provided that m+n=1 or 2.

[3] The method according to the above [1] or [2], wherein an amino acid sequence of the κ chain variable region-binding peptide comprises an amino acid sequence having a sequence identity of 85% or more with any one of amino acid sequences of SEQ ID NOs: 12 to 20, may optionally comprise a peptide reside having 1 or more and 15 or less amino acid residues at at least one of an N-terminal side and a C-terminal side, and has the cysteine residue at at least one of the N-terminal or the C-terminal.

[4] The method according to the above [1], wherein the κ chain variable region-binding peptide except for the terminal cysteine residue is a multimer of κ chain variable region-binding domains.

[5] The method according to any one of the above [1] to [4], wherein a water-insoluble carrier having a maleimide group on a surface is used as the water insoluble carrier, and the ligand is immobilized through the maleimide group.

[6] An affinity separation matrix,

comprising a water-insoluble carrier and a κ chain variable region-binding peptide as a ligand,

wherein the κ chain variable region-binding peptide has a cysteine residue at at least one of an N-terminal and a C-terminal, and

the κ chain variable region-binding peptide is immobilized on the water-insoluble carrier through the terminal cysteine residue.

[7] The affinity separation matrix according to the above [6], wherein an amino acid sequence of the κ chain variable region-binding peptide comprises an amino acid sequence of the following formula (I):

(X¹)ₘ—(Y¹)ₚ-Glu-X²-Val-Thr-Ile-Lys-X³-Asn-X⁴—X⁵—X⁶—X⁷—X⁸-Gly-X⁹—X¹⁰-Gln-X¹¹-Ala-X¹²-Phe-Lys-Gly-Thr-Phe-X¹³—X¹⁴-Ala-X¹⁵—X¹⁶—X¹⁷-Ala-Tyr-X¹⁸-Tyr-Ala-X¹⁹—X²⁰-Leu-X²¹-Lys-X²²—X²³-Gly-X²⁴-Tyr-Thr-X²⁵-Asp-X²⁶—X²⁷-Asp-X²⁸-Gly-X²⁹-Thr-X³⁰-Asn-Ile-X³¹-Phe-Ala-Gly-(Y²)_{q}—(X³²)ₙ (I)

wherein X¹ is Cys, X² is Gln or Glu, X³ is Glu, Ala or Val, X⁴ is Ile or Leu, X⁵ is Tyr or Ile, X⁶ is Phe or Tyr, X⁷ is Ala or Glu, X⁸ is Asp or Asn, X⁹ is Thr, Ser or Lys, X¹⁰ is Val, Ile or Thr, X¹¹ is His, Thr or Asn, X¹² is Thr or Glu, X¹³ is Ala or Glu, X¹⁴ is Glu or Lys, X¹⁵ is Thr or Val, X¹⁶ Ala or Ser, X¹⁷ is Glu, Asp or Lys, X¹⁸ is Arg or Ala, X¹⁹ is Asp or Asn, X²⁰ is Leu, Thr or Ala, X²¹ is Ser, Lys or Ala, X²² is Glu, Asp or Val, X²³ His or Asn, X²⁴ is Lys or Glu, X²⁵ is Ala or Val, X²⁶ is Leu or Val, X²⁷ is Glu or Ala, X²⁸ is Gly or Lys, X²⁹ is Tyr, Leu or Asn, X³⁰ is Ile or Leu, X³¹ is Arg or Lys, X³² is Cys, Y¹ and Y² are independently peptide residues having 1 or more and 15 or less amino acid residues, m, n, p and q are independently 0 or 1, provided that m+n=1 or 2.

[8] The affinity separation matrix according to the above [6] or [7], wherein an amino acid sequence of the κ chain variable region-binding peptide comprises an amino acid sequence having a sequence identity of 85% or more with any one of amino acid sequences of SEQ ID NOs: 12 to 20, may optionally comprise a peptide reside having 1 or more and 15 or less amino acid residues at at least one of an N-terminal side and a C-terminal side, and has the cysteine residue at at least one of the N-terminal or the C-terminal.

[9] The affinity separation matrix according to the above [6], wherein the κ chain variable region-binding peptide except for the terminal cysteine residue is a multimer of κ chain variable region-binding domains.

[10] The affinity separation matrix according to any one of the above [6] to [9], wherein the water-insoluble carrier has a maleimide group on a surface, and the terminal cysteine residue is bound to the maleimide group.

[11] A method for producing a peptide containing a κ chain variable region, comprising the steps of:

contacting a liquid sample containing the peptide containing the κ chain variable region with the affinity separation matrix according to any one of the above [6] to [10], and

separating the peptide containing the κ chain variable region on the affinity separation matrix from the affinity separation matrix.

The affinity separation matrix of one or more embodiments of the present invention is produced by immobilizing a κ chain variable region-binding peptide having a cysteine residue at the end on a water-insoluble carrier. A general peptide is randomly immobilized on a water-insoluble carrier, since a general peptide has many reactive functional groups at the side chain, such as a hydroxy group and an amino group in addition to a thiol group. On the one hand, since the κ chain variable region-binding peptide used in one or more embodiments of the present invention has a cysteine residue at the end and the terminal cysteine residue is not only inherently very reactive but also hardly sterically hindered, the cysteine residue may be especially reactive and preferentially reacted with a reactive group on a water-insoluble carrier. As a result, the κ chain variable region-binding peptide used in one or more embodiments of the present invention is considered to be orientationally immobilized on a water-insoluble carrier. With respect to the affinity separation matrix of one or more embodiments of the present invention, therefore, the ligand density is clearly improved and further the binding capacity to a κ chain variable region-containing peptide per the ligand density is also increased. Thus, the affinity separation matrix of one or more embodiments of the present invention is very useful for purifying a κ chain variable region-containing peptide.

Since the affinity separation matrix for a κ chain variable region-containing peptide has an excellent binding capacity to a κ chain variable region-containing peptide of an immunoglobulin, the affinity separation matrix also exhibits an excellent binding capacity to not, only a general antibody but also an antibody fragment which does not have an Fc region but has a κ chain variable region, such as tab and scFv. Accordingly, when the affinity separation matrix of one or more embodiments of the present invention is used, an antibody fragment drug can be efficiently purified. Recently, an antibody fragment drug has been actively developed, since such a drug can be produced at low cost. One or more embodiments of the present invention are industrially very useful, since one or more embodiments of the present invention can contribute to the practical use of an antibody fragment drug.

### BRIEF DESCRIPTION OF THE DRAWINGS

The FIGURE is a photograph of an electrophoresis gel to demonstrate the result of the purification of Fab from a solution containing the Fab and an impurity by using the affinity separation matrix of one or more embodiments of the present invention.

### DETAILED DESCRIPTION OF THE EMBODIMENTS

Hereinafter, first, the method for producing the affinity separation matrix of one or more embodiments of the present invention is described.

1. Method for Producing Affinity Separation Matrix of One or More Embodiments of the Present Invention

The method for producing the affinity separation matrix according to one or more embodiments of the present invention comprises the step of immobilizing a κ chain variable region-binding peptide having a cysteine residue at either an N-terminal or a C-terminal or at both of an N-terminal and a C-terminal as a ligand on a water-insoluble carrier through the terminal cysteine residue. Hereinafter, “κ chain variable region” is abbreviated as “VL-κ” in some cases.

The term “ligand” in one or more embodiments of the present invention means a substance and a functional group to selectively bind to a target molecule to be captured from an aggregate of molecules on the basis of a specific affinity between molecules, such as binding between an antigen and an antibody, and in one or more embodiments of the present invention, means the peptide which specifically binds to VL-κ of an immunoglobulin. In one or more embodiments of the present invention, the term “ligand” also means an “affinity ligand”. In addition, “affinity” and “binding property” are used synonymously with “affinity force” and “binding force”.

The VL-κ-binding peptide used in one or more embodiments of the present invention is not particularly restricted as long as the peptide has a cysteine residue at either of an N-terminal or a C-terminal or at both of an N-terminal and a C-terminal, and has a binding property to VL-κ. For example, as the VL-κ-binding peptide, Protein L having a binding property to VL-κ and a part of Protein L, such as a domain of Protein L, may be used.

The term “peptide” in one or more embodiments of the present invention means any molecules having a polypeptide structure. In the range of the “peptide”, not only a so-called protein but also a fragmented protein and a protein to which other peptide is bound through a peptide bond are included. The term “domain” means a unit of higher-order structure of a protein. A domain is composed of from dozens to hundreds of amino acid residues and means a peptide unit which can sufficiently serve some kind of a physicochemical or biochemical function. The term “variant” of a protein or peptide means a protein or peptide obtained by introducing at least one substitution, addition or deletion of an amino acid into a sequence of a wild protein or peptide. The number of a mutation may be preferably not more than 20 or not more than 15, more preferably not more than 10 or not more than 8, and even more preferably not more than 5 or not more than 3.

The “Protein L” is a protein derived from a cell wall of anaerobic gram-positive coccus in the genus of Peptostreptococcus. Hereinafter, “Protein L” is abbreviated as “PpL” in some cases. PpL may be preferably derived from Peptostreptococcus magnus, and preferably two kinds of PpL derived from Peptostreptococcus magnus 312 strain and Peptostreptococcus magnus 3316 strain but is not restricted thereto (Non-patent documents 4 to 6). In the present disclosure, in some cases, the PpL derived from Peptostreptococcus magnus 312 strain is abbreviated as “PpL 312”, and the PpL derived from Peptostreptococcus magnus 3316 strain is abbreviated as “PpL 3316”. The amino acid sequence of PpL 312 is shown as SEQ ID NO: 1, and the amino acid sequence of PpL 3316 is shown as SEQ ID NO: 2. The amino acid sequences of SEQ ID NOs: 1 and 2 also contain a signal sequence. As shown in SEQ ID NOs: 1 and 2, the sequence of the original PpL does not contain cysteine.

PpL contains a plurality of VL-κ-binding domains having 70 to 80 residues in the protein molecule. The number of VL-κ-binding domains contained in PpL 312 is 5, and the number of VL-κ-binding domains contained in PpL 3316 is 4. The VL-κ-binding domains contained in PpL 312 are referred to as B1 domain (SEQ ID NO: 3), B2 domain (SEQ ID NO: 4), B3 domain (SEQ ID NO: 5), B4 domain (SEQ ID NO: 6), and B5 domain (SEQ ID NO: 7) in the order from the N-terminal, and the VL-κ-binding domains contained in PpL 3316 are referred to as C1 domain (SEQ ID NO: 8), C2 domain (SEQ ID NO: 9), C3 domain (SEQ ID NO: 10), and C4 domain (SEQ ID NO: 11) in the order from the N-terminal (Non-patent documents 5 and 6). The amino acid sequence of the VL-κ-binding peptide according to one or more embodiments of the present invention may be preferably an amino acid sequence based on SEQ ID NOs: 3 to 11.

It has been found from a research that about 20 residues of the VL-κ-binding domain of PpL at the N-terminal part do not form a specific secondary structure; and even when the N-terminal part is deleted, the three-dimensional structure and the VL-κ-binding property of the VL-κ-binding domain are maintained (Non-patent Document 7). For example, peptides having the amino acid sequence of SEQ ID NO: 12 with respect to B1 domain, the amino acid sequence of SEQ ID NO: 13 with respect to B2 domain, the amino acid sequence of SEQ ID NO: 14 with respect to B3 domain, the amino acid sequence of SEQ ID NO: 15 with respect to B4 domain, the amino acid sequence of SEQ ID NO: 16 with respect to B5 domain, the amino acid sequence of SEQ ID NO: 17 with respect to C1 domain, the amino acid sequence of SEQ ID NO: 18 with respect to C2 domain, the amino acid sequence of SEQ ID NO: 19 with respect to C3 domain, and the amino acid sequence of SEQ ID NO: 20 with respect to C4 domain also function as a VL-κ-binding domain. The amino acid sequence of the VL-κ-binding peptide according to one or more embodiments of the present invention may also be preferably an amino acid sequence based on SEQ ID NOs: 12 to 20.

The peptide having an amino acid sequence of SEQ ID NOs: 1 and 2 with deletion of several residues at the N-terminal and/or the C-terminal is considered to have a VL-κ-binding property. The number of residues to be deleted may be preferably 1 or more and 5 or less, more preferably 1 or more and 4 or less, even more preferably 1 or more and 3 or less, even more preferably 1 or 2, and even more preferably 1.

PpL is a protein in which 4 or 5 VL-κ-binding domains are linked in tandem as described above. The VL-κ-binding peptide of one or more embodiments of the present invention, therefore, may be a monomer or a multimer composed of the 2 or more, preferably 3 or more, even more preferably 4 or more, and even more preferably 5 or more VL-κ-binding peptides as monodomains, as one embodiment. With respect to the upper limit of the number of the domains to be linked, 10 or less is exemplified, 8 or less may be preferred, and 6 or less may be more preferred. The multimer may be a homomultimer in which one kind of the VL-κ-binding peptides are linked, such as homodimer and homotrimer, or a heteromultimer in which two or more kinds of the VL-κ-binding domains are linked, such as heterodimer and heterotrimer. As described above, either an N-terminal or a C-terminal or both of an N-terminal and a C-terminal are cysteine residues.

A method for connecting monomer VL-κ-binding peptides in the above-described multimer is exemplified by a connecting method through one or more amino acid residues but is not restricted thereto. The number of the amino acid residue for connection is not particularly restricted, and may be preferably 20 residues or less, and more preferably 15 residues or less. It may be preferred to use a sequence to link B1 and B2, B2 and B3, B3 and B4, B4 and B5, C1 and C2, C2 and C3, or C3 and C4 of wild PpL. From another point of view, it may be preferred that the amino acid residue for connection does not destabilize a three dimensional structure of the monomer VL-κ-binding peptide.

As one of the embodiments, a fusion peptide characterized in that the VL-κ-binding peptide or a multimer composed of the linked 2 or more peptides is fused as one component with other peptide having a different function is exemplified as the ligand of the affinity separation matrix according to one or more embodiments of the present invention. Such a fusion peptide is exemplified by a peptide fused with albumin or GST (glutathione S-transferase) but is not restricted to the examples. In addition, peptides fused with a nucleic acid such as DNA aptamer, a drug such as an antibiotic or a polymer such as PEG (polyethylene glycol) are also included in one or more embodiments of the present invention as long as the fused component is available for the affinity separation matrix of one or more embodiments of the present invention.

Since either of ends of the VL-κ-binding peptide may be a cysteine residue in one or more embodiments of the present invention, the terminal amino acid residue of the above-described PpL or a part thereof may be substituted by cysteine or a cysteine residue may be further added to the end. In addition, a peptide which is not involved in VL-κ-binding property and of which terminal residue is cysteine may be further added to the end of PpL or a part thereof. The length of such a peptide containing the cysteine to be added is not particularly restricted and may be exemplified by 2 or more and 20 or less residues, preferably 2 or more and 15 or less residues, more preferably 2 or more and 10 or less residues, even more preferably 2 or more and 5 or less residues, and even more preferably 2 residues.

The VL-κ-binding peptide used in one or more embodiments of the present invention as a ligand has a cysteine residue at at least one of an N-terminal and a C-terminal. Either of an N-terminal or a C-terminal may be a cysteine residue, or both of an N-terminal and a C-terminal may be cysteine residues. In order to improve the orientation on a water-insoluble carrier more surely, it may be preferred that only either of an N-terminal or a C-terminal is a cysteine residue, and it may be more preferred that only a C-terminal is a cysteine residue.

The VL-κ-binding peptide used in one or more embodiments of the present invention is exemplified by a peptide represented by the formula (I), and a peptide which has an amino acid sequence having a sequence identity of 85% or more with any one of the amino acid sequences of SEQ ID NO: 12 to 20, which may optionally have a peptide residue of 1 or more and 15 or less amino acid residues at either of an N-terminal side or a C-terminal side or at both of the terminal sides and which has a cysteine residue at either of an N-terminal or a C-terminal or at both of the terminals. Hereinafter, the peptide represented by the formula (I) is abbreviated as “the peptide (I)” in some cases. The amino acid sequence of the peptide (I) is an amino acid sequence corresponding the amino acid sequence of SEQ ID NO: 21 to which a cysteine residue added at either of an N-terminal or a C-terminal or at both of the terminals, or the amino acid sequence of SEQ ID NO: 21 to which a peptide residue of 1 or more and 15 or less amino acid residues is added at an N-terminal side and/or a C-terminal side and to which a cysteine residue added at either of an N-terminal or a C-terminal or at both of the terminals. The number of the peptide residue may be preferably not more than 12 or not more than 10, more preferably not more than 8 and not more than 5, and even more preferably 1 or 2. The above-described sequence identity may be preferably not less than 86%, not less than 88% or not less than 90%, more preferably not less than 92%, not less than 94% or not less than 95%, even more preferably not less than 96%, not less than 98% or not less than 99%, and particularly preferably not less than 99.5% or not less than 99.8%. The term “sequence identity” in one or more embodiments of the present invention means an identity degree of an amino acid between 2 or more amino acid sequences. When an identity between certain two amino acid sequences is higher, an identity and a homology between the sequences are higher. Whether 2 kinds of amino acid sequences show a specific identity or not can be analyzed by directly comparing the sequences, specifically by using a program for amino acid sequence multiple alignment, such as Clustal (http://www.clustal.org/omega/), and a commercially available sequence analysis software.

The ratio of the binding force of the VL-κ-binding peptide according to one or more embodiments of the present invention to a VL-κ-containing peptide to the binding constant of the peptide having the amino acid sequence of SEQ ID NO: 16 as a comparison object may be preferably included in a range of 0.01 times or more and 100 times or less, more preferably 0.02 times or more and 50 times or less, and preferably 0.1 times or more and 10 times or less.

The VL-κ-binding peptide used in one or more embodiments of the present invention can be prepared by an ordinary method. Specifically, the DNA which encodes an amino acid sequence of the desired VL-κ-binding peptide or a fragment thereof is chemically synthesized, the DNA encoding the VL-κ-binding peptide is amplified by PCR, and the DNA is incorporated into a vector. Escherichia coli or the like is infected with the obtained vector and cultivated. The VL-κ-binding peptide may be purified from the cultivated bacterial body or the culture medium by chromatography or the like. Alternatively, cysteine may be chemically bound to at either of an N-terminal or a C-terminal or at both of the terminals of the VL-κ-binding peptide of which terminal residue is not cysteine.

The “insoluble carrier” used in one or more embodiments of the present invention shows insolubility to an aqueous solvent as a solvent of the liquid sample containing a VL-κ-containing peptide, and when the ligand is immobilized on the insoluble carrier, the peptide which specifically binds to the ligand can be purified. The insoluble carrier usable in one or more embodiments of the present invention is exemplified by an inorganic carrier such as glass beads and silica gel; an organic carrier composed of a synthetic polymer such as cross-linked polyvinyl alcohol, cross-linked polyacrylate, cross-linked polyacrylamide and cross-linked polystyrene; an organic carrier composed of a polysaccharide such as crystalline cellulose, cross-linked cellulose, cross-linked agarose and cross-linked dextran; and a composite carrier obtained by the combination of the above carriers, such as an organic-organic composite carrier and an organic-inorganic composite carrier. The commercially available product thereof is exemplified by porous cellulose gel GCL2000, Sephacryl S-1000 prepared by crosslinking allyl dextran and methylene bisacrylamide through a covalent bond, an acrylate carrier Toyopearl, a cross-linked agarose carrier Sepharose CL4B, and a cross-linked cellulose carrier Cellufine. It should be noted, however, that the insoluble carrier usable in one or more embodiments of the present invention is not restricted to the carriers exemplified as the above.

It may be preferred that the insoluble carrier usable in one or more embodiments of the present, invention has large surface area and is porous with a large number of fine pores having a suitable size in terms of a purpose and a method of the use of the affinity separation matrix. The carrier may have any form such as beads, monolith, fiber and film including hollow fiber, and any form can be selected.

As a method for immobilizing the VL-κ-binding peptide as the ligand on a water-insoluble carrier in one or more embodiments of the present invention, an ordinary method may be used. At least, the VL-κ-binding peptide is immobilized on a water-insoluble carrier through a terminal cysteine residue.

Specifically, there is a reactive group such as an amino group, a hydroxy group and a carboxy group on the surface of a general water-insoluble carrier. The reactive group may be activated or substituted by other reactive group, or a linker group having a reactive group may be introduced on the reactive group. For example, when an epoxy group is introduced on the surface of a water-insoluble carrier by using epichlorohydrin, diglycidyl ether, 1,4-bis(2,3-epoxypropoxy)butane or the like, or when an iodoacetyl group, a bromoacetyl group or the like is introduced on the surface of a water-insoluble carrier, a coupling reaction between the VL-κ-binding peptide and the reactive group can be easily accelerated. Since the VL-κ-binding peptide used in one or more embodiments of the present invention has a cysteine residue having a highly reactive thiol group at the end and the thiol group is hardly sterically hindered, it is thought that the VL-κ-binding peptide is bound to a water-insoluble carrier mainly through the thiol group and is immobilized on the water-insoluble carrier with high orientation.

In particular, it may be preferred in one or more embodiments of the present invention that a water-insoluble carrier which has a maleimide group on the surface is used and the VL-κ-binding peptide is immobilized by using the maleimide group. Since a maleimide group is selectively reacted with a thiol group, the VL-κ-binding peptide can be immobilized with much higher orientation by using a water-insoluble carrier having a maleimide group on the surface. As a result, a ligand density and a binding ability to a VL-κ-containing peptide can be increased more surely.

When a linker group is used for immobilizing the ligand on a water-insoluble carrier, the linker group is not particularly restricted. The linker group is exemplified by a C₁₋₆ alkylene group, an amino group (—NH—), an imino group (>C═N— or —N═C<), an ether group (—O—), a thioether group (—S—), a carbonyl group (—C(═O)—), a thionyl group (—C(═S)—), an ester group (—C(═O)—O— or —O—C(═O)—), an amide group (—C(═O)—NH— or —NH—C(═O)—), a sulfoxide group (—S(═O)—), a sulfonyl group (—S(═O)₂—), a sulfonylamide group (—NH—S(═O)₂— and —S(═O)₂—NH—), and a group formed by binding a plurality of the above-described groups. When the linker group is formed by binding a plurality of the above-described groups, the number of the bound groups may be preferably not more than 10 or not more than 5, and more preferably 3 or less.

A spacer molecule composed of a plurality of atoms may be introduced between the ligand and carrier. Alternatively, the ligand may be directly immobilized on the carrier. In addition, the VL-κ-binding peptide of one or more embodiments of the present invention may be chemically modified for immobilization.

2. Affinity Separation Matrix of One or More Embodiments of the Present Invention

In the affinity separation matrix according to one or more embodiments of the present invention produced by the above-described production method, the VL-κ-binding peptide is immobilized on a water-insoluble carrier through the terminal cysteine residue at the end of the peptide with high orientation and high ligand density. Thus, since the affinity separation matrix of one or more embodiments of the present invention has a high affinity for a VL-κ-containing peptide, the matrix is very useful for purifying a VL-κ-containing peptide.

Specifically, one or more embodiments of the present invention are characterized in that the VL-κ-binding peptide in the affinity separation matrix has cysteine having a thiol group used for an immobilization reaction at the N-terminal or C-terminal, and the peptide is immobilized on a water-insoluble carrier through the side chain thiol group of the terminal cysteine. Though there may be lysine having an amino group in a side chain and threonine having a hydroxy group in a side chain in the sequence of the VL-κ-binding peptide, which an amino group and a hydroxy group may be reactive to the immobilization, the VL-κ-binding peptide can be immobilized with controlling an orientation due to the immobilization through the thiol group in the side chain of the terminal cysteine, and thus, the ligand can efficiently bind to a VL-κ-containing peptide. As a result, the affinity separation matrix of one or more embodiments of the present invention exhibits a higher binding capacity to a VL-κ-containing peptide in comparison with a carrier on which PpL, which does not originally have cysteine, is immobilized.

As long as the VL-κ-binding peptide having a cysteine residue at the end is immobilized on a water-insoluble carrier with controlling orientation and a binding capacity to the VL-κ-binding peptide is improved in comparison with the water-insoluble carrier on which PpL not having cysteine is immobilized, a part of the peptides may be immobilized on a carrier through a group except for a thiol group, such as an amino group in the side chain of lysine and a hydroxy group in the side chain of threonine. In addition, it is permissible that a part of the VL-κ-binding peptide may be immobilized on a water-insoluble carrier through the N-terminal amino group as long as a binding capacity to the VL-κ-binding peptide as a property of the prepared affinity separation matrix is improved in comparison with an affinity separation matrix on which PpL not having cysteine is immobilized.

An “immunoglobulin (Ig)” is a glycoprotein produced by a B cell of a lymphocyte and has a function to recognize a specific molecule such as a protein to be bound. An immunoglobulin has not only a function to specifically bind to a specific molecule referred to as antigen but also a function to detoxify and remove an antigen-containing factor in cooperation with other biological molecule or cell. An immunoglobulin is generally referred to as “antibody”, and the name is inspired by such functions.

All of immunoglobulins basically have the same molecular structure. The basic structure of an immunoglobulin is a Y-shaped four-chain structure. The four-chain structure is composed of two light chains and two heavy chains of polypeptide chains. A light chain (L chain) is classified into two types of λ chain and κ chain, and all of immunoglobulins have either of the chains. A heavy chain (H chain) is classified into five types of γ chain, μ chain, α chain, δ chain and ε chain, and an immunoglobulin is classified into an isotype depending on the kind of a heavy chain. An immunoglobulin G (IgG) is a monomer immunoglobulin, is composed of two γ chains and two light chains and has two antigen-binding sites.

A lower half vertical part in the “Y” shape of an immunoglobulin is referred to as a “Fc region”, and an upper half “V” shaped part is referred to as a “Fab region”. An Fc region has an effector function to initiate a reaction after an antibody binds to an antigen, and a Fab region has a function to bind to an antigen. A Fab region of a heavy chain and an Fc region are bound to each other through a hinge part. Papain, which is a proteolytic enzyme and which is contained in papaya, decomposes a hinge part to cut into two Fab regions and one Fc region. The part close to the tip of the “Y” shape in a Fab region is referred to as a “variable region (V region)”, since there are various changes of the amino acid sequence in order to bind to various antigens. A variable region of a light chain is referred to as a “VL region”, and a variable region of a heavy chain is referred to as a “VH region”. A Fab region except for a V region and an Fc region are referred to as a “constant region (C region)”, since there is relatively less change. A constant region of a light chain is referred to as a “CL region”, and a constant region of a heavy chain is referred to as a “CH region”. A CH region is further classified into three regions of CH1 to CH3. A Fab region of a heavy chain is composed of a VH region and CH1, and an Fc region of a heavy chain is composed of CH2 and CH3. There is a hinge part between CH1 and CH2. PpL binds to a variable region of which light chain is κ chain (VL-κ) (Non-patent Documents 5 to 7).

The VL-κ-binding peptide used as the ligand of the affinity separation matrix according to in one or more embodiments of the present invention is based on the sequence of Protein L (PpL) and binds to a κ chain variable region (VL-κ) of an immunoglobulin. A VL-κ-containing protein to which the affinity separation matrix of one or more embodiments of the present invention binds contains at least VL-κ and may be IgG containing a Fab region and an Fc region without deficiency, or other Ig series such as IgM, IgD and IgA, or a derivative of an immunoglobulin molecule prepared by a protein engineering mutation. An immunoglobulin molecule derivative to which the VL-κ-binding affinity separation matrix of one or more embodiments of the present invention binds is not particularly restricted as long as the derivative contains VL-κ. For example, the immunoglobulin molecule derivative is exemplified by a Fab fragment prepared by fragmenting immunoglobulin G into a Fab region only, scFv and diabody consisting of only a variable region of immunoglobulin G, chimeric immunoglobulin G prepared by replacing a part of human immunoglobulin G domains with an immunoglobulin G domain of other organism to be fused, immunoglobulin G of which sugar chain in the Fc region is mutated, and a scFv fragment to which a drug is covalently bound.

A value of a ligand density of an affinity separation matrix is calculated by dividing an amount of a ligand immobilized on the affinity separation matrix by a volume of the affinity separation matrix. A volume of an affinity separation matrix used for calculating a ligand density means a volume of a matrix on which the ligand is immobilized, which can bind to a VL-κ-containing peptide to be retained and which is in a condition of a gel. For example, the volume is measured after the affinity separation matrix of one or more embodiments of the present invention is dispersed in water, a neutral phosphate buffer or the like, the obtained dispersion is added into a measuring tool such as a measuring cylinder, and then the measuring tool is sufficiently left to stand until the apparent volume is not decreased any more.

Depending on the material of the matrix, it may take a long time to leave the matrix to stand still. In such a case, the volume can be measured after the measuring tool is slightly tapped until the apparent volume is not decreased any more and then left to stand still. Since a commercially available pre-packed carrier is prepared by filling a column with a predetermined volume amount of a matrix, the predetermined volume is defined as the volume of the matrix.

The mass of the ligand immobilized on the affinity separation matrix can be calculated as the difference between the mass of the ligand originally used for reacting with a water-insoluble carrier and the mass of the ligand which is not immobilized and which is recovered after the immobilization reaction. The masses of the ligands may be directly measured or indirectly measured by absorbance measurement or the like. For example, the mass of the ligand immobilized on the affinity separation matrix can be determined by calculating the ligand amount in a ligand solution to be used for reacting with a water-insoluble carrier by absorbance measurement, calculating the unreacted ligand amount by absorbance measurement of the unreacted ligand solution after the immobilization reaction, and calculating the difference between the amounts. The ligand amount can also be determined by using an absorbance coefficient calculated from an amino acid sequence. When the ligand amount can be directly measured, the ligand amount can be determined by preparing a solution thereof, obtaining an absorbance coefficient of the solution, and using the absorbance coefficient.

Alternatively, the mass of the ligand immobilized on the affinity separation matrix can be determined by protein quantitation method using bicinchoninic acid (BCA) reagent. For example, an amount of the immobilized ligand per measured volume of the affinity separation matrix can be determined by dispersing the affinity separation matrix in water, adding the obtained dispersion into a measuring tool such as a measuring cylinder, measuring the volume after the measuring tool is sufficiently left to stand still until the apparent volume is not decreased any more, further mixing BCA reagent to be reacted for a predetermined time, and measuring an absorbance at 562 nm. The mass of the ligand on this occasion can be determined by preliminarily measuring an absorbance value at 562 nm which is dependent on the mass of the ligand. The method for determining the ligand density is not restricted to the above-described example.

For example, the affinity of the VL-κ-binding peptide as the ligand of the affinity separation matrix according to one or more embodiments of the present invention for a VL-κ-containing peptide can be determined by Biacore system (GE Healthcare) utilizing a surface plasmon resonance principle or a biosensor such as Octet system (Pall ForteBio) utilizing BioLayer Interferometry method but is not restricted thereto.

A binding parameter to evaluate an affinity for a VL-κ-containing peptide is exemplified by an association constant (K_{A}) and a dissociation constant (K₀) (Nagata et al., “Real-time analysis experiment of biomaterial interactions”, Springer-Verlag Tokyo, 1998, page 41).

A binding capacity of the affinity separation matrix according to one or more embodiments of the present invention for a VL-κ-containing peptide can be represented as, for example, static binding capacity. A static binding capacity corresponds to the maximum binding capacity of an affinity separation matrix itself and is not dependent on a flow rate or the like. In one or more embodiments of the present invention, for example, the binding capacity to Fab is compared while 55% DBC (Dynamic Binding Capacity) is selected as quasi-static binding capacity and Fab is selected as a VL-κ-containing peptide.

With respect to a method for evaluating the above-described 55% DBC, for example, a column is filled with a predetermined amount of the affinity separation matrix, the column is equilibrated, and then a solution of a VL-κ-containing peptide is flown at a constant flow rate 55% DBC can be determined by determining a total amount of a VL-κ-containing peptide in a solution which is flown until an absorbance of the eluted VL-κ-containing peptide solution exceeds 55% of an absorbance of the flown VL-κ-containing peptide solution and dividing the determined total amount by the amount of the affinity separation matrix.

When a value of 55% DBC is higher, a binding capacity to a VL-κ-containing peptide is higher. A value of 55% DBC may be preferably 20 mg/mL-gel or more, more preferably not less than 25 mg/mL-gel or not less than 30 mg/mL-gel, and even more preferably 50 mg/mL-gel or more. The term “1 mL-gel” which is a volume of the affinity separation matrix as a standard means 1 mL of a volume of the affinity separation matrix measured by tapping or leaving to stand the affinity separation matrix in a state of suspension and gel until the volume does not become decreased any more.

3. Preparation Method of VL-κ-Containing Peptide

As described above, the affinity separation matrix of one or more embodiments of the present invention has a very high affinity for a VL-κ-containing peptide. Thus, a VL-κ-containing peptide can be purified by using the affinity separation matrix of one or more embodiments of the present invention. Hereinafter, each step of the method for producing a VL-κ-containing peptide according to one or more embodiments of the present invention is described.

Step 1: Step for Adsorbing VL-κ-Containing Peptide

In the present step, a VL-κ-containing peptide is adsorbed on the water-insoluble carrier by contacting the affinity separation matrix of one or more embodiments of the present invention with a liquid sample containing the VL-κ-containing peptide.

The above-described liquid sample is not particularly restricted as long as the liquid sample contains a VL-κ-containing peptide to be purified, and it may be preferred that the liquid sample is a solution in which a VL-κ-containing peptide is dissolved in water. Such a liquid sample is exemplified by a serum sample which contains a VL-κ-containing peptide, a culture medium or a homogenate supernatant of a bacterium and a fungus which culture medium and supernatant contain a VL-κ-containing peptide, and a homogenate of a hybridoma which produces a monoclonal antibody. In addition, it may be preferred that the liquid sample is approximately neutral, and the pH value thereof is 6 or more and 8 or less. The solvent of the liquid sample may be water only, may contain a water-miscible organic solvent such as C₁₋₄ alcohol as long as the liquid sample contains water as a main component, and may be a buffer solution of which pH is 6 or more and 8 or less.

In the present step 1, for example, a VL-κ-containing peptide is selectively adsorbed on the VL-κ-binding peptide by filling a column with the affinity separation matrix of one or more embodiments of the present invention to obtain an affinity column and flowing the liquid sample through the affinity column.

Step 2: Step for Washing Affinity Separation Matrix

In the present step, the affinity separation matrix on which a VL-κ-containing peptide is adsorbed in the above-described Step 1 is washed to remove an impurity except for the VL-κ-containing peptide. Even after the present step, a VL-κ-containing peptide is adsorbed on the affinity separation matrix in the column. The affinity separation matrix of one or more embodiments of the present invention is excellent in the absorption and retention performance of a VL-κ-containing peptide from the step of adding a liquid sample through the step of washing the matrix, since the matrix has high affinity for a VL-κ-containing peptide.

As a washing liquid usable for washing the affinity separation matrix in the present Step 2, a washing liquid which does not disturb interaction between a VL-κ-containing peptide and the VL-κ-binding peptide is used. For example, a buffer of which pH is 5 or more and 8 or less can be used as the washing liquid, but the kind of a washing liquid and an additive agent are not particularly restricted as long as the VL-κ-containing peptide is not leaked from the matrix due to the washing liquid.

Step 3: Step for Separating VL-κ-Containing Peptide

In the present step, a VL-κ-containing peptide is separated from the affinity separation matrix on which the VL-κ-containing peptide is adsorbed by using an acidic buffer. By the present Step 3, a purified VL-κ-containing peptide can be obtained.

In the present Step 3, the pH of an acidic buffer used for separating a VL-κ-containing peptide from the affinity separation matrix may be appropriately adjusted, and for example, can be adjusted to about 2.0 or more and 4.0 or less. Into the acidic buffer used for eluting a VL-κ-containing peptide, a substance for promoting dissociation from the matrix may be added.

Step 4: Step for Regenerating Affinity Separation Matrix

In the present step, the affinity separation matrix from which a VL-κ-containing peptide is separated in the above-described Step 3 is regenerated by washing with an alkaline aqueous solution. It is not needed to necessarily perform the present Step 4 after the above-described Step 3, and the present step may be performed once every three iterations of the above Step 1 to Step 3, once every five iterations, or once every ten iterations. Specifically, when a performance of the affinity separation matrix, such as binding capacity, is maintained, the present step is not necessarily performed. The implementation frequency and condition of the present step are different depending on the liquid sample containing a VL-κ-containing peptide.

The “alkaline aqueous solution” usable for the regeneration of the affinity separation matrix means an aqueous solution which exhibits alkalinity to the extent that a purpose such as washing and sterilization can be achieved. For example, a sodium hydroxide aqueous solution of not less than 0.01 M and not more than 1.0 M or not less than 0.01 N and not more than 1.0 N can be used. The pH of the alkaline aqueous solution may be preferably about 12 or more and 14 or less.

The time to treat the affinity separation matrix by an alkaline aqueous solution after the above-described Step 3 is not particularly restricted and may be appropriately adjusted, since a damage degree of the peptide is different depending on the concentration of the alkaline aqueous solution and the temperature at the treatment. For example, when the concentration of sodium hydroxide is 0.05 M and the temperature during immersion is atmospheric temperature, the lower limit of the time to immerse the affinity separation matrix into the alkaline aqueous solution may be preferably 1 hour, more preferably 2 hours, more preferably 4 hours, more preferably 10 hour, and more preferably 20 hours, but is not particularly restricted as long as the affinity separation matrix can be regenerated.

The affinity separation matrix of one or more embodiments of the present invention is very excellent in affinity for a VL-κ-containing peptide. Thus, by using the affinity separation matrix of one or more embodiments of the present invention, a VL-κ-containing peptide can be efficiently purified from a liquid sample containing the VL-κ-containing peptide; as a result, the VL-κ-containing peptide can be efficiently produced.

The present application claims the benefit of the priority date of Japanese patent application No. 2016-95264 filed on May 11, 2016. All of the contents of the Japanese patent application No. 2016-95264 filed on May 11, 2016, are incorporated by reference herein.

### EXAMPLES

Hereinafter, one or more embodiments of the present invention are described in more detail with Examples; however, the present invention is not restricted to the following Examples.

The κ chain variable region-binding peptide used in the following Examples is described as “peptide name+sequence number−amino acid residue at the C-terminal”. For example, the κ chain variable region-binding peptide prepared by adding cysteine at the C-terminal of the amino acid sequence of SEQ ID NO: 22 is described as “PpL22-C”, and the κ chain variable region-binding peptide prepared by adding lysine at the C-terminal of the amino acid sequence of SEQ ID NO: 22 is described as “PpL22-K”. Hereinafter, a “κ chain variable region” is abbreviated as “VL-κ”.

### Example 1

### Preparation of VL-κ-Binding Peptide-Immobilized Carrier by Using Epoxy-Activated Carrier

(1) Preparation Various Expression Plasmid for VL-κ-Binding Peptide

The peptide of SEQ ID NO: 24 (“PpL22-C”) was designed by linking the 4 VL-κ-binding domains having the amino acid sequence of SEQ ID NO: 23 through the amino acid sequence which linked between VL-κ-binding domains having the amino acid sequence of SEQ ID NO: 1 contained in Protein L derived from Peptostreptococcus magnus 312 strain to obtain the amino acid sequence of SEQ ID NO: 22 and adding cysteine at the C-terminal. A base sequence of SEQ ID NO: 25 encoding the peptide was designed by reverse translation from the amino acid sequence of SEQ ID NO: 24. The DNA (SEQ ID NO: 26) which had the base sequence (SEQ ID NO: 25), PstI recognition site at 5′ end and XbaI recognition site at 3′ end was synthesized as an artificial synthetic gene by outsourcing to Eurofins Genomics K. K. The expression plasmid after subcloning was digested by restriction enzyme PstI and XbaI manufactured by Takara Bio Inc. to obtain a DNA fragment. An expression vector pNCMO2 for Brevibacillus Expression System manufactured by Takara Bio Inc. was digested by the same restriction enzyme, and the obtained DNA fragment was ligated thereto to obtain an expression vector into which DNA encoding the amino acid sequence of PpL22-C was inserted. The ligation reaction was performed by using “Ligation high” manufactured by TOYOBO CO., LTD. in accordance with the protocol attached to the product, and Escherichia coli JM109 strain manufactured by Takara Bio Inc. was used for preparing the plasmid. The base sequence of each expression plasmid DNA was confirmed by using DNA sequencer 3130×1 Genetic Analyzer manufactured by Applied Biosystems. A sequencing PCR of each plasmid DNA was performed by using BigDye Terminator v.1.1 Cycle Sequencing Kit manufactured by Applied Biosystems in accordance with the protocol attached to the product, and the obtained sequencing product was purified by using a plasmid purification kit (“BigDye XTerminator Purification Kit” manufactured by Applied Biosystems) in accordance with the protocol attached to the product to be used for sequence analysis.

(2) Preparation of VL-κ-Binding Peptide

Brevibacillus choshinensis SP3 strain manufactured by Takara Bio Inc. was transformed by the obtained plasmid, and the genetically modified bacterium which produced and secreted PpL22-C was cultivated The genetically modified bacterium was cultivated in 30 mL of A culture medium (polypeptone 3.0%, yeast extract 0.5%, glucose 3%, magnesium sulfate 0.01%, ferric sulfate 0.001%, manganese chloride 0.001%, zinc chloride 0.0001%) containing 60 μg/mL of neomycin with shaking at 30° C. for 3 days. After the cultivation, the culture medium was subjected to centrifugation at 15,000 rpm and at 25° C. for 5 minutes to remove the bacterial body.

PpL22-C was purified from the obtained culture supernatant by cation exchange chromatography for which a column (“Tricorn 10/200” manufactured by GE Healthcare Bioscience) was filled with a cation exchange carrier (“UnoSphere S” manufactured by Bio-Rad). Specifically, sodium acetate was added to the culture supernatant at a final concentration of 50 mM, and the pH thereof was adjusted to 4.0 by acetic acid. The cation exchange column was equilibrated by buffer A for cation exchange (50 mM CH₃COOH—CH₃COONa, pH 4.0), and the culture supernatant was added into the column. After the column was washed by the buffer A for cation exchange, PpL22-C was eluted to be obtained by using salt concentration gradient with the buffer A for cation exchange and buffer B for cation exchange (50 mM CH₃COOH—CH₃COONa, 1 M NaCl, pH 4.0). Then, the PpL22-C was purified by anion exchange chromatography for which a column (“Tricorn 10/200” manufactured by GE Healthcare Bioscience) was filled with an anion exchange carrier (“Nuvia Q” manufactured by Bio-Rad). Specifically, the obtained PpL22-C solution was dialyzed by using a buffer A for anion exchange (50 mM Tris-HCl, pH 8.0). The anion exchange column was equilibrated by buffer A for anion exchange, and the solution was added into the equilibrated column. After the column was washed by the buffer A for anion exchange, PpL22-C was eluted to be obtained by using salt concentration gradient with the buffer A for anion exchange and buffer B for anion exchange (50 mM Tris-HCl, 1.0 M NaCl, pH 8.0). The obtained PpL22-C was dialyzed by using ultrapure water, and an aqueous solution containing the PpL22-C only was obtained as a final purified sample. The above-described protein purification by chromatography with a column was performed by using “AKTAavant 25 system” manufactured by GE Healthcare Bioscience.

(3) Preparation of Epoxy-Activated Carrier

Highly-crosslinked crystalline cellulose which was manufactured by JNC and which was a gel obtained by the method described in JP 2009-242770 A or US 20090062118 A was used as a raw material water-insoluble cellulose carrier.

On a glass filter, 2 mL of gelatinous carrier in a wet state was transferred and washed with 10 mL of ultrapure water 3 times. The washed carrier was transferred into a centrifuge tube, and the predetermined amount of 1,4-bis(2,3-epoxypropoxy)butane was added thereto. The mixture was stirred at 37° C. for 30 minutes. Then, 9.2 M sodium hydroxide aqueous solution was added so that the final concentration became 1 M. The mixture was stirred at 37° C. for 2 hours. The carrier was transferred on a glass filter, and the reaction mixture was removed by reduced pressure. The carrier on the glass filter was washed by 30 mL of ultrapure water to obtain an epoxidized carrier.

(4) Immobilization of VL-κ-Binding Peptide on Epoxy-Activated Carrier

The PpL22-C which was prepared in the above-described Example 1(2) and which had cysteine at the C-terminal was reduced by using 100 mM DTT, and was further pre-treated by using a desalting column (“HiTrap Desalting” manufactured GE Healthcare) to remove DTT and exchange the liquid part by a coupling buffer.

On a glass filter, 1.2 mL of the epoxy-activated carrier obtained in the above-described Example 1(3) was transferred and washed with ultrapure water and 1.5 mL of a coupling buffer (150 mM NaH₂PO₄, 1 mM EDTA, pH 9.5) 3 times. Then, the epoxy-activated carrier was transferred into a centrifuge tube, and the reduced PpL22-C was added thereto. The mixture was reacted at 37° C. for 30 minutes. After the reaction, sodium sulfate powder was added thereto so that the final concentration became 0.8 M. After the addition of sodium sulfate, the mixture was reacted at 37° C. for 2 hours. After the reaction, the carrier was transferred on a glass filter and washed by 5 mL of an immobilization buffer to recover unreacted PpL22-C. Then, after the carrier was washed with 5 mL of ultrapure water 3 times, the carrier was washed with 5 mL of an inactivating buffer containing thioglycerol (200 mM NaHCO₃, 100 mM NaCl, 1 mM EDTA, pH 8.0) 3 times. After the carrier was collected by dispersing in the inactivating buffer containing thioglycerol, the carrier was transferred into a centrifuge tube for a reaction at 25° C. overnight. Then, the carrier was transferred on a glass filter, and washed with ultrapure water and 5 mL of a washing buffer (100 mM Tris-HCl, 150 mM NaCl, pH 8.0) 3 times. The carrier was transferred into a centrifuge tube and stirred at 25° C. for 20 minutes. The carrier was transferred on a glass filter and washed with 5 mL of ultrapure water 3 times. The carrier was further washed with 10 mL of ultrapure water and 10 mL of 20% ethanol, and then collected by dispersing in 20% ethanol.

The absorbance of the recovered unreacted PpL22-C at 280 nm was measured by using a spectrometer, an amount of the unreacted VL-κ-binding peptide was determined on the basis of an absorbance coefficient calculated from the amino acid sequence, and the ligand densities of a weight and a mole number were calculated. The ligand density of PpL22-C in the prepared affinity separation matrix is shown in Table 1.

### Example 2

### Preparation of VL-κ-Binding Peptide-Immobilized Carrier by Using Epoxy-Activated Carrier

Similarly to the above-described Example 1, the affinity separation matrix was prepared. On the affinity separation matrix, the VL-κ-binding peptide having the variant peptide amino acid sequence of SEQ ID NO: 27 added cysteine at the C-terminal (PpL27-C) was immobilized. The ligand density of PpL27-C in the prepared affinity separation matrix is shown in Table 1.

### Example 3

### Preparation of VL-κ-Binding Peptide-Immobilized Carrier by Using Maleimide-Activated Carrier

(1) Preparation of Maleimide-Activated Carrier

A commercially available NHS-activated carrier (“NHS Activated Sepharose 4 Fast Flow” manufactured by GE Healthcare Bioscience) was used as a raw material water-insoluble carrier. On a glass filter, 1.5 mL of gelatinous carrier in a wet state was transferred. After isopropanol as a preservative liquid was removed by suction, the carrier was washed with 5 mL of 1 mM ice-cooled hydrochloric acid. Then, after the carrier was washed with 5 mL of a coupling buffer (20 mM NaH₂PO₄—Na₂PO₄, 150 mM sodium chloride, pH 7.2), the carrier was collected with dispersing in the coupling buffer and transferred into a centrifuge tube. A 10 mM solution prepared by dissolving N-[ε-maleimidecaproic acid]hydrazine.TFA (EMCH, manufactured by Thermo Fisher Scientific) in the coupling buffer was added to the centrifuge tube with the carrier for the reaction at 25° C. for 1 hour. Then, the carrier was transferred on a glass filter, washed with 10 mL of washing buffer A (0.5 M ethanolamine, 0.5 M sodium chloride, pH 7.2), 10 mL of the coupling buffer and 10 mL of the washing buffer A in this order, and left to stand still at 25° C. for 15 minutes. The carrier was further washed with 10 mL of the coupling buffer. By the above procedure, a maleimide group was bound to the carrier.

(2) Immobilization of VL-κ-Binding Peptide on Maleimide-Activated Carrier

Next, PpL22-C was immobilized on the maleimide-activated carrier. Before PpL22-C was used for immobilization, PpL22-C was reduced by using 100 mM DTT and was further pre-treated by using a desalting column (“HiTrap Desalting” manufactured GE Healthcare) to remove DTT and exchange the liquid part by the coupling buffer.

The maleimide-activated carrier was transferred into a centrifuge tube, and a PpL22-C solution was further added thereto for the reaction at 25° C. for 2 hours. Then, the reacted carrier was transferred on a glass filter, and the unreacted PpL22-C was recovered by washing with 7 mL of the coupling buffer. Next, after the carrier was washed with 10 mL of a washing buffer B (50 mM L-cysteine, 100 mM NaH₂PO₄—Na₂HPO₄, 0.5 M sodium chloride, pH 7.2), 10 mL of the coupling buffer and 10 mL of the washing buffer B in this order, the carrier was left to stand still at 25° C. for 15 minutes. After the carrier was further washed with 10 mL of the coupling buffer, 10 mL of ultrapure water and 10 mL of 20% ethanol, the carrier was dispersed in 20% ethanol to be collected to obtain PpL22-C-immobilized affinity separation matrix.

The absorbance of the recovered unreacted PpL22-C at 280 nm was measured by using a spectrometer, and an amount of the unreacted PpL22-C was determined on the basis of an absorbance coefficient calculated from the amino acid sequence. An amount of the immobilized PpL22-C was calculated on the basis of the difference of the originally used PpL22-C and determined amount of the unreacted PpL22-C. Further, the ligand density was calculated on the basis of the volume of the carrier. The ligand density of PpL22-C in the prepared affinity separation matrix is shown in Table 1.

### Example 4

### Preparation of VL-κ-Binding Peptide-Immobilized Carrier by Using Maleimide-Activated Carrier

Similarly to the above-described Example 3, an affinity separation matrix on which PpL27-C was immobilized as a VL-κ-binding peptide was prepared. The ligand density of PpL27-C in the prepared affinity separation matrix is shown in Table 1.

### Comparative Example 1

Similarly to the above-described Example 1(1) and (2), PpL22-K which had the amino acid sequence corresponding to SEQ ID NO: 22 having lysine at the C-terminal was prepared. On a glass filter, 1.2 mL of the epoxy-activated carrier obtained in the above-described Example 1(3) was transferred and washed with ultrapure water and 1.5 mL of a coupling buffer (150 mM NaH₂PO₄, 1 mM EDTA, pH 8.5) 3 times. Then, the epoxidized carrier was transferred into a centrifuge tube, and PpL22-K was added thereto. The mixture was reacted at 37° C. for 30 minutes. After the reaction, sodium sulfate powder was added thereto so that the final concentration became 1.0 M. After adding sodium sulfate, the mixture was reacted at 37° C. for 5 hours. After the reaction, the carrier was transferred on a glass filter and washed with 5 mL of the immobilization buffer 3 times to recover the unreacted PpL22-K. Then, the carrier was washed with 5 mL of ultrapure water 3 times and then 5 mL of an inactivating buffer containing thioglycerol (200 mM NaHCO₃, 100 mM NaCl, 1 mM EDTA, pH 8.0) 3 times. The carrier was collected by dispersing in the inactivating buffer containing thioglycerol, and then transferred into a centrifuge tube for reaction at 25° C. overnight. Then, the carrier was transferred on a glass filter and washed with ultrapure water and 5 mL of a washing buffer (100 mM Tris-HCl, 150 mM NaCl, pH 8.0) 3 times. The carrier was transferred into a centrifuge tube and stirred at 25° C. for 20 minutes. The carrier was transferred on a glass filter and washed with 5 mL of ultrapure water 3 times. After the carrier was washed with 10 mL of ultrapure water and 10 mL of 20% ethanol, the carrier was collected by dispersing in 20% ethanol.

The absorbance of the recovered unreacted PpL22-K at 280 nm was measured by using a spectrometer, an amount of the unreacted PpL22-K was determined on the basis of an absorbance coefficient calculated from the amino acid sequence, and a ligand density was calculated. The ligand density of PpL22-K in the prepared affinity separation matrix is shown in Table 1.

TABLE 1

| Carrier | | | Ligand | Ligand |
|---|---|---|---|---|
| | Surface | | density | density |
| | functional | | (mg/mL- | (μmol/mL- |
| | group | Ligand | gel) | gel) |
| Comparative | Epoxy group | PpL22 - K | 5.8 | 169.5 |
| example 1 | | | | |
| Example 1 | Epoxy group | PpL22 - C | 5.4 | 157.9 |
| Example 2 | Epoxy group | PpL27 - C | 5.1 | 149.7 |
| Example 3 | Maleimide group | PpL22 - C | 12.5 | 365.2 |
| Example 4 | Maleimide group | PpL27 - C | 10.1 | 295.1 |

### Test Example 1

### Evaluation of Affinity for VL-κ-Containing Peptide

(1) Preparation of Fab Fragment Derived from IgG (IgG-Fab)

A Fab fragment was selected as a VL-κ-containing peptide. Humanized monoclonal IgG product having VL-κ as a raw material was fragmented into a Fab fragment and an Fc fragment by using papain, and the Fab fragment only was purified. Hereinafter, a method for producing Fab derived from anti-IgE monoclonal antibody (general name: “omalizumab”) is described, and basically other monoclonal Fab can be also produced a similar method.

Specifically, in the case of anti IgE monoclonal antibody, “Xolair” manufactured by Novartis AG was dissolved as a humanized monoclonal antibody IgG drug product in a buffer for papain digestion (0.1 M AcOH—AcONa, 2 mM EDTA, 1 mM cysteine, pH 5.5). Agarose on which papain was immobilized (“Papain Agarose from papaya latex” manufactured by SIGMA) added thereto. The mixture was incubated at 37° C. for about 8 hours while the mixture was stirred by a rotator. The reaction mixture containing both of Fab fragment and Fc fragment was separated from the agarose on which papain was immobilized. A Fab solution was obtained as a flow-through fraction by affinity chromatography using KANEKA KanCapA™ column manufactured by KANEKA CORPORATION from the reaction mixture. The obtained Fab solution was subjected to purification by gel filtration chromatography using Superdex 75 10/300 GL column to obtain a Fab solution. A standard buffer was used for equilibration and separation. The above protein purification by chromatography was performed by using AKTAavant 25 system.

(2) Evaluation of Affinity of Various VL-κ-Binding Peptide for Fab

The affinity of PpL22-C obtained in Example 1(2) and PpL27-C obtained in Example 2 as VL-κ-binding peptides for Fab was analyzed by using a biosensor Biacore 3000 (manufactured by GE Healthcare Bioscience) utilizing surface plasmon resonance. In the test present Example, the VL-κ-binding peptide was immobilized on a sensor tip, and the VL-κ obtained in Test example 1(1) was flown on the tip to detect the interaction between the two. The VL-κ-binding peptide was immobilized on a sensor tip CM5 by amine coupling method using N-hydroxysuccinimide (NHS) and N-ethyl-N′-(3-dimethylaminopropyl)carbodiimide hydrochloride (EDC), and ethanolamine was used for blocking. All of the sensor tip and reagents for immobilization were manufactured by GE Healthcare Bioscience. The VL-κ-binding peptide solution was diluted to about 10 times by using an immobilization buffer (10 mM CH₃COOH—CH₃CONa, pH 4.5), and the VL-κ-binding peptide was immobilized on the sensor tip in accordance with the protocol attached to the Biacore 3000. In addition, a reference cell as negative control was also prepared by activating another flow cell on the tip with EDC/NHS and then immobilizing ethanoliamine. The Fab was dissolved in a running buffer (20 mM NaH₂PO₄—Na₂HPO₄, 150 mM NaCl, 0.005% P-20, pH 7.4) to prepare a protein solution having a concentration in the range of 1 to 10000 nM, and each protein solution was added to the sensor tip at a flow rate of 40 μL/min for 60 seconds. Binding response curves at the time of addition (association phase, for 60 seconds) and after the addition (dissociation phase, for 60 seconds) were sequentially obtained at a measurement temperature of 25° C. After each measurement, 25 mM NaOH was added for 30 seconds to remove the added Fab remaining on the sensor chip and to regenerate the sensor chip. With respect to the obtained binding response curve from which the binding response curve of the reference cell was subtracted, a fitting analysis was conducted by 1:1 binding model using a software included in the system, “BIA evaluation”, and an association constant (K_{A}=kₒₙ/k_{off}) for human IgG was calculated. As a result, it was confirmed that the K_{A} values (M⁻¹) of PpL22-C and PpL27-C were on the order of 10⁶, and both of PpL22-C and PpL27-C had similar affinities for the Fab used for the evaluation. It is thought that the K_{A} value of PpL22-K is similar to that of PpL22-C, since the difference between PpL22-K and PpL22-C is only one amino acid at the C-terminal.

### Test Example 2

### Evaluation of Binding Capacity of VL-κ-Binding Peptide-Immobilized Carrier to Fab

With respect to the affinity separation matrix prepared in Examples 1 to 4 and Comparative example 1, the binding capacity to the VL-κ-containing Fab prepared in Test example 1(1) was evaluated. In addition, for the comparison with a commercially available product, commercially available Protein L carrier (“HiTrap Protein L” manufactured by GE Healthcare, 1 mL-gel) was similarly evaluated as Comparative example 2. The Fab prepared in Test example 1(1) was dissolved in an equilibrating buffer (20 mM NaH₂PO₄—Na₂HPO₄, 150 mM sodium chloride, pH 7.4) in a concentration of 1 mg/mL, and the solution was used.

A column (“Tricorn 5/50 column” GE Healthcare Bioscience) was filled with 1 mL-gel of the affinity separation matrix and connected to chromatography system AKTAavant 25. The column was equilibrated by flowing 3 CV of an equilibrating buffer (20 mM NaH₂PO₄—Na₂HPO₄, 150 mM sodium chloride, pH 7.4) at a flow rate of 0.25 mL/min. Then, the Fab solution was flown at a flow rate of 0.25 mL/min until a monitoring absorbance exceeded 55% of 100% Abs₂₈₀. Next, 10 CV of the equilibrating buffer was flown at a flow rate of 0.25 mL/min, and subsequently 3 CV of an elution buffer (50 mM citric acid, pH 2.5) was flown to elute the Fab. The total amount of the Fab solution which was flown until a monitoring absorbance exceeded 55% of 100% Abs₂₈₀ was defined as 55% DBC, i.e. quasi-static binding capacity, to Fab. Since the commercially available column product as Comparative example 2 was preliminarily filled with Protein L carrier, the column was directly connected to the chromatography system and the similar evaluation was performed. The measurement result is shown in Table 2. The ligand density of Comparative example 2 described in Table 2 was that described in the instruction manual attached to the product.

TABLE 2

| Carrier | | | |
|---|---|---|---|
| | Surface | | |
| | functional | Ligand density | 55% DBC |
| | group | (mg/mL-gel) | (mg/mL-gel) |
| Comparative | Epoxy group | 5.8 | 19.1 |
| example 1 | | | |
| Comparative | Unknown | 10 | 21.7 |
| example 2 | | | |
| Example 1 | Epoxy group | 5.4 | 31.4 |
| Example 2 | Epoxy group | 5.1 | 23.8 |
| Example 3 | Maleimide group | 12.5 | 109.4 |
| Example 4 | Maleimide group | 10.1 | 85.7 |

TABLE 3

| | Ligand density | 55% DBC | 55% DBC/ |
|---|---|---|---|
| Carrier | (μmol/mL-gel) | (μmol/mL-gel) | Ligand density |
| Comparative | 169.5 | 382.0 | 2.3 |
| example 1 | | | |
| Example 1 | 157.9 | 628.0 | 4.0 |
| Example 2 | 149.7 | 476.0 | 3.2 |
| Example 3 | 365.2 | 2188.0 | 6.0 |
| Example 4 | 295.1 | 1714.0 | 5.8 |

As the result shown in Table 2, 55% DBC of the affinity separation matrixes of Examples 1 and 2 to Fab were higher than that of Comparative example 1. In addition, though the ligand densities of Examples 1 and 2 were lower than that of Comparative example 2 by about 50%, 55% DBC of Examples 1 and 2 were similar to or higher than that of Comparative example 2. Furthermore, 55% DBC of Examples 3 and 4 were much higher than those of Comparative examples 1 and 2. It was demonstrated from the results that the affinity separation matrix according to one or more embodiments of the present invention has an excellent property.

In addition, ligand density and 55% DBC or the basis of mole number and 55% DBC per ligand density are shown in Table 3 for comparison. Since the molecular weight of the ligand of Comparative example 2 was not disclosed, the result of Comparative example 2 is not shown in Table 3. It was confirmed from the result shown in Table 3 that 55% DBC per ligand density of the affinity separation matrixes of Examples 1 to 4 containing the VL-κ-binding peptide as a ligand having cysteine at the C-terminal side was significantly higher than that of Comparative example 1 containing the VL-κ-binding peptide as a ligand not having cysteine at the C-terminal side. This experimental result is considered to be attributable to the fact that the ligand is immobilized on a water-insoluble carrier through cysteine with controlling the orientation and the ligand can be efficiently bound to Fab due to the existence of cysteine at the C-terminal of the ligand. The 55% DBC value per ligand density of Examples 3 and 4 was much higher than that of Examples 1 and 2. This experimental result is considered to be attributable to the fact that the ligand of Examples 3 and 4 was immobilized on a water-insoluble carrier mainly at the terminal thiol group only and the orientation of the ligand was remarkably controlled, since the active group of the water-insoluble carrier used in Example 3 and 4 was a maleimide group, while a side chain thiol group, a side chain hydroxy group and a side chain amino group in the peptide were also reacted in addition to the terminal thiol group in Examples 1 and 2, since the active group of the water-insoluble carrier used in Examples 1 and 2 is an epoxy group; as a result, in Examples 3 and 4, the orientation of the ligand was remarkably controlled and the binding efficiency of the ligand to VL-κ was remarkably improved.

### Example 3

### Purification of Fab Contained in Culture Supernatant of Escherichia coli

It was confirmed whether Fab in a solution containing an impurity can be purified or not by using the carrier prepared in Example 2. As the solution containing an impurity, a cell homogenization liquid of E. coli was used. Specifically, E. coli (“HB101” manufactured by Takara Bio Inc.) was transformed by using pUC-series plasmid, and the transformant was cultivated in 2YT culture medium at 37° C. overnight. Then, the cell was collected, the collected cell was disrupted by using a sonicator, and the obtained mixture was subjected to centrifugation to obtain a supernatant as an impurity-containing solution. In the obtained impurity-containing solution, Fab was added so that the final concentration became 0.25 mg/mL to be used for the following measurement.

An empty column (“Tricorn 5/50 column” manufactured by GE Healthcare) was filled with the carrier prepared in the above-described Example 2 and connected to chromatography system AKTAavant 25. The column was equilibrated by flowing 5 CV of an equilibrating buffer (20 mM NaH₂PO₄—Na₂HPO₄, 150 mM sodium chloride, pH 7.4). Then, 40 mL of the above-described solution containing Fab and an impurity was flown. Then, the carrier was washed by flowing 10 CV of the equilibrating buffer, and subsequently Fab was eluted by flowing 10 CV of an eluting buffer (50 mM citric acid, pH 3.0). Further, after 3 CV of the equilibrating buffer was flown, 5 CV of a strong washing buffer (50 mM citric acid, pH 2.5) was flown. Finally, the purification was finished by flowing 5 CV of the equilibrating buffer. The flow rate in all of the steps was adjusted to 0.25 mL/min. The solutions of sample addition fraction, washing fraction and elution fraction were respectively collected and used for SDS-PAGE analysis. The solution of the elution step was reutilized to be used by using a NaOH solution.

The collected solutions described above were subjected to SDS-PAGE in a non-reducing condition by using a mini PAGE system with a built-in power supply (“PageRun” manufactured by ATTO CORPORATION) and 15% polyacrylamide precast gel (“e-PAGEL” manufactured by ATTO CORPORATION) in accordance with the attached manual. The photograph of the electrophoresis gel after staining treatment and decoloring treatment is shown as the FIGURE.

As shown the FIGURE, it was confirmed that Fab was not leaked, since there was not a Fab component in the fraction at the time of the addition of the solution containing Fab and an impurity to the carrier and the washing fraction. In addition, it was confirmed that highly pure Fab can be obtained in the elution fraction. It was demonstrated from the result that highly pure Fab can be obtained by one step by the purification using the affinity separation matrix produced by one or more embodiments of the present invention. Furthermore, the affinity separation matrix of one or more embodiments of the present invention may be practically used for separating and purifying an antibody fragment containing VL-κ.

Although the disclosure has been described with respect to only limited number of embodiments, those skilled in the art, having benefit of this disclosure, will appreciate that various other embodiments may be devised without departing from the scope of the present invention. Accordingly, the scope of the invention should be limited only by the attached claims.

What is claimed is:

1. A method for producing an affinity separation matrix, the method comprising:
immobilizing a κ chain variable region-binding peptide on a water-insoluble carrier through a terminal cysteine residue of the κ chain variable region-binding peptide,
wherein the cysteine residue is located at an N-terminal or a C-terminal of the κ chain variable region-binding peptide,
wherein the κ chain variable region-binding peptide is a ligand having an affinity for a κ chain variable region, and
wherein the affinity separation matrix comprises the ligand and the water-insoluble carrier.

2. The method according to claim 1, wherein an amino acid sequence of the κ chain variable region-binding peptide comprises the following amino acid sequence:
(X¹)ₘ—(Y¹)ₚ-Glu-X²-Val-Thr-Ile-Lys-X³-Asn-X⁴—X⁵—X⁶—X⁷—X⁸-Gly-X⁹—X¹⁰-Gln-X¹¹-Ala-X¹²-Phe-Lys-Gly-Thr-Phe-X¹³—X¹⁴-Ala-X¹⁵—X¹⁶—X¹⁷-Ala-Tyr-X¹⁸-Tyr-Ala-X¹⁹—X²⁰-Leu-X²¹-Lys-X²²—X²³-Gly-X²⁴-Tyr-Thr-X²⁵-Asp-X²⁶—X²⁷-Asp-X²⁸-Gly-X²⁹-Thr-X³⁰-Asn-Ile-X³¹-Phe-Ala-Gly-(Y²)_{q}—(X³²)ₙ,
wherein X¹ is Cys, X² is Gln or Glu, X³ is Glu, Ala or Val, X⁴ is Ile or Leu, X⁵ is Tyr or Ile, X⁶ is Phe or Tyr, X⁷ is Ala or Glu, X⁸ is Asp or Asn, X⁹ is Thr, Ser or Lys, X¹⁰ is Val, Ile or Thr, X¹¹ is His, Thr or Asn, X¹² is Thr or Glu, X¹³ is Ala or Glu, X¹⁴ is Glu or Lys, X¹⁵ is Thr or Val, X¹⁶ is Ala or Ser, X¹⁷ is Glu, Asp or Lys, X¹⁸ is Arg or Ala, X¹⁹ is Asp or Asn, X²⁰ is Leu, Thr or Ala, X²¹ is Ser, Lys or Ala, X²² is Glu, Asp or Val, X²³ is His or Asn, X²⁴ is Lys or Glu, X²⁵ is Ala or Val, H²⁶ is Leu or Val, X²⁷ is Glu or Ala, X²⁸ Gly or Lys, X²⁹ is Tyr, Leu or Asn, X³⁰ is Ile or Leu, X³¹ is Arg or Lys, X³² is Cys, Y¹ and Y² are each independently 1 to 15 amino acid residues, and m, n, p and q are each independently 0 or 1, provided that m+n=1 or 2.

3. The method according to claim 1,
wherein an amino acid sequence of the κ chain variable region-binding peptide comprises an amino acid sequence having a sequence identity of 85% or more with an amino acid sequence selected from the group consisting of SEQ ID NOs: 12 to 20, and
wherein the κ chain variable region-binding peptide comprises the cysteine residue located at the N-terminal or the C-terminal.

4. The method according to claim 3, wherein the κ chain variable region-binding peptide further comprises 1 to 15 amino acid residues at at least one of an N-terminal side or a C-terminal side.

5. The method according to claim 1, wherein the κ chain variable region-binding peptide except for the terminal cysteine residue is a multimer of κ chain variable region-binding domains.

6. The method according to claim 1, wherein the water-insoluble carrier has a maleimide group on a surface, and the ligand is immobilized through the maleimide group.

7. An affinity separation matrix, comprising:
a water-insoluble carrier; and
a ligand,
wherein the ligand is a κ chain variable region-binding peptide,
wherein the κ chain variable region-binding peptide comprises a cysteine residue at an N-terminal or a C-terminal, and
wherein the κ chain variable region-binding peptide is immobilized on the water-insoluble carrier through the terminal cysteine residue.

8. The affinity separation matrix according to claim 7, wherein an amino acid sequence of the κ chain variable region-binding peptide comprises the following amino acid sequence:
(X¹)ₘ—(Y¹)ₚ-Glu-X²-Val-Thr-Ile-Lys-X³-Asn-X⁴—X⁵—X⁶—X⁷—X⁸-Gly-X⁹—X¹⁰-Gln-X¹¹-Ala-X¹²-Phe-Lys-Gly-Thr-Phe-X¹³—X¹⁴-Ala-X¹⁵—X¹⁶—X¹⁷-Ala-Tyr-X¹⁸-Tyr-Ala-X¹⁹—X²⁰-Leu-X²¹-Lys-X²²—X²³-Gly-X²⁴-Tyr-Thr-X²⁵-Asp-X²⁶—X²⁷-Asp-X²⁸-Gly-X²⁹-Thr-X³⁰-Asn-Ile-X³¹-Phe-Ala-Gly-(Y²)_{q}—(X³²)ₙ,
wherein X¹ is Cys, X² is Gln or Glu, X³ is Glu, Ala or Val, X⁴ is Ile or Leu, X⁵ is Tyr or Ile, X⁶ is Phe or Tyr, X⁷ is Ala or Glu, X⁸ is Asp or Asn, X⁹ is Thr, Ser or Lys, X¹⁰ is Val, Ile or Thr, X¹¹ is His, Thr or Asn, X¹² is Thr or Glu, X¹³ is Ala or Glu, X¹⁴ is Glu or Lys, X¹⁵ is Thr or Val, X¹⁶ is Ala or Ser, X¹⁷ is Glu, Asp or Lys, X¹⁸ is Arg or Ala, X¹⁹ is Asp or Asn, X²⁰ is Leu, Thr or Ala, X²¹ is Ser, Lys or Ala, X²² is Glu, Asp or Val, X²³ is His or Asn, H²⁴ Lys or Glu, X²⁵ is Ala or Val, X²⁶ is Leu or Val, X²⁷ is Glu or Ala, X²⁸ is Gly or Lys, X²⁹ is Tyr, Leu or Asn, X³⁰ is Ile or Leu, X³¹ is Arg or Lys, X³² is Cys, Y¹ and Y² are each independently 1 to 15 amino acid residues, and m, n, p and q are each independently 0 or 1, provided that m+n=1 or 2.

9. The affinity separation matrix according to claim 7, wherein an amino acid sequence of the κ chain variable region-binding peptide comprises an amino acid sequence having a sequence identity of 85% or more with an amino acid sequence selected from the group consisting of SEQ ID NOs: 12 to 20,
wherein the κ chain variable region-binding peptide comprises the cysteine residue located at the N-terminal or the C-terminal.

10. The affinity separation matrix according to claim 9, wherein the κ chain variable region-binding peptide further comprises 1 to 15 amino acid residues at at least one of an N-terminal side or a C-terminal side.

11. The affinity separation matrix according to claim 7, wherein the κ chain variable region-binding peptide except for the terminal cysteine residue is a multimer of κ chain variable region-binding domains.

12. The affinity separation matrix according to claim 7, wherein the water-insoluble carrier has a maleimide group on a surface, and the terminal cysteine residue is bound to the maleimide group.

13. A method for producing a peptide containing a κ chain variable region, comprising:
contacting a liquid sample containing the peptide containing the κ chain variable region with the affinity separation matrix according to claim 7, and
separating the peptide containing the chain variable region on the affinity separation matrix from the affinity separation matrix.


SEQUENCE LISTING (27 sequences)

SEQ ID NO: 1 - protein, 719 aa, Peptostreptococcus magnus
MAALAGAIVVTGGVGSYAADEPIDLEKLEEKRDKENVGNLPKFDNEVKDGSENPMAKYPDFDDEASTRFETENNEFEEKKVVSDNFFDQSEHPFVENKEETPETPETDSEEEVTIKANLIFANGSTQTAEFKGTFEKATSEAYAYADTLKKDNGEYTVDVADKGYTLNIKFAGKEKTPEEPKEEVTIKANLIYADGKTQTAEFKGTFEEATAEAYRYADALKKDNGEYTVDVADKGYTLNIKFAGKEKTPEEPKEEVTIKANLIYADGKTQTAEFKGTFEEATAEAYRYADLLAKENGKYTVDVADKGYTLNIKFAGKEKTPEEPKEEVTIKANLIYADGKTQTAEFKGTFAEATAEAYRYADLLAKENGKYTADLEDGGYTINIRFAGKKVDEKPEEKEQVTIKENIYFEDGTVQTATFKGTFAEATAEAYRYADLLSKEHGKYTADLEDGGYTINIRFAGKEEPEETPEKPEVQDGYASYEEAEAAAKEALKNDDVNKSYTIRQGADGRYYYVLSPVEAEEEKPEAQNGYATYEEAEAAAKKALENDPINKSYSIRQGADGRYYYVLSPVEAETPEKPVEPSEPSTPDVPSNPSNPSTPDVPSTPDVPSNPSTPEVPSNPSTPGNEEKPGNEQKPGNEQKPGNEQKPGNEQKPGNEQKPDQPSKPEKEENGKGGVDSPKKKEKAALPKAGSEAEILTLAAASLSSVAGAFISLKKRK

SEQ ID NO: 2 - protein, 992 aa, Peptostreptococcus magnus
MKINKKLLMAALAGAIVVGGGANAYAAEEDNTDNNLSMDEISDAYFDYHGDVSDSVDPVEEEIDEALAKALAEAKETAKKHIDSLNHLSETAKKLAKNDIDSATTINAINDIVARADVMERKTAEKEEAEKLAAAKETAKKHIDELKHLADKTKELAKRDIDSATTINAINDIVARADVMERKTAEKEEAEKLAAAKETAKKHIDELKHLADKTKELAKRDIDSATTIDAINDIVARADVMERKLSEKETPEPEEEVTIKANLIFADGSTQNAEFKGTFAKAVSDAYAYADALKKDNGEYTVDVADKGLTLNIKFAGKKEKPEEPKEEVTIKVNLIFADGKTQTAEFKGTFEEATAKAYAYADLLAKENGEYTADLEDGGNTINIKFAGKETPETPEEPKEEVTIKVNLIFADGKIQTAEFKGTFEEATAKAYAYANLLAKENGEYTADLEDGGNTINIKFAGKETPETPEEPKEEVTIKVNLIFADGKTQTAEFKGTFEEATAEAYRYADLLAKVNGEYTADLEDGGYTINIKFAGKEQPGENPGITIDEWLLKNAKEEAIKELKEAGITSDLYFSLINKAKTVEGVEALKNEILKAHAGEETPELKDGYATYEEAEAAAKEALKNDDVNNAYEIVQGADGRYYYVLKIEVADEEEPGEDTPEVQEGYATYEEAEAAAKEALKEDKVNNAYEVVQGADGRYYYVLKIEDKEDEQPGEEPGENPGITIDEWLLKNAKEDAIKELKEAGISSDIYFDAINKAKTVEGVEALKNEILKAHAEKPGENPGITIDEWLLKNAKEAAIKELKEAGITAEYLFNLINKAKTVEGVESLKNEILKAHAEKPGENPGITIDEWLLKNAKEDAIKELKEAGITSDIYFDAINKAKTIEGVEALKNEILKAHKKDEEPGKKPGEDKKPEDKKPGEDKKPEDKKPGEDKKPEDKKPGKTDKDSPNKKKKAKLPKAGSEAEILTLAAAALSTAAGAYVSLKKRK

SEQ ID NO: 3 - protein, 76 aa, Peptostreptococcus magnus
KEETPETPETDSEEEVTIKANLIFANGSTQTAEFKGTFEKATSEAYAYADTLKKDNGEYTVDVADKGYTLNIKFAG

SEQ ID NO: 4 - protein, 72 aa, Peptostreptococcus magnus
KEKTPEEPKEEVTIKANLIYADGKTQTAEFKGTFEEATAEAYRYADALKKDNGEYTVDVADKGYTLNIKFAG

SEQ ID NO: 5 - protein, 72 aa, Peptostreptococcus magnus
KEKTPEEPKEEVTIKANLIYADGKTQTAEFKGTFEEATAEAYRYADLLAKENGKYTVDVADKGYTLNIKFAG

SEQ ID NO: 6 - protein, 72 aa, Peptostreptococcus magnus
KEKTPEEPKEEVTIKANLIYADGKTQTAEFKGTFAEATAEAYRYADLLAKENGKYTADLEDGGYTINIRFAG

SEQ ID NO: 7 - protein, 73 aa, Peptostreptococcus magnus
KKVDEKPEEKEQVTIKENIYFEDGTVQTATFKGTFAEATAEAYRYADLLSKEHGKYTADLEDGGYTINIRFAG

SEQ ID NO: 8 - protein, 71 aa, Peptostreptococcus magnus
KETPEPEEEVTIKANLIFADGSTQNAEFKGTFAKAVSDAYAYADALKKDNGEYTVDVADKGLTLNIKFAGK

SEQ ID NO: 9 - protein, 71 aa, Peptostreptococcus magnus
KEKPEEPKEEVTIKVNLIFADGKTQTAEFKGTFEEATAKAYAYADLLAKENGEYTADLEDGGNTINIKFAG

SEQ ID NO: 10 - protein, 74 aa, Peptostreptococcus magnus
KETPETPEEPKEEVTIKVNLIFADGKIQTAEFKGTFEEATAKAYAYANLLAKENGEYTADLEDGGNTINIKFAG

SEQ ID NO: 11 - protein, 75 aa, Peptostreptococcus magnus
KETPETPEEPKEEVTIKVNLIFADGKTQTAEFKGTFEEATAEAYRYADLLAKVNGEYTADLEDGGYTINIKFAGK

SEQ ID NO: 12 - protein, 63 aa, Peptostreptococcus magnus
EEVTIKANLIFANGSTQTAEFKGTFEKATSEAYAYADTLKKDNGEYTVDVADKGYTLNIKFAG

SEQ ID NO: 13 - protein, 63 aa, Peptostreptococcus magnus
EEVTIKANLIYADGKTQTAEFKGTFEEATAEAYRYADALKKDNGEYTVDVADKGYTLNIKFAG

SEQ ID NO: 14 - protein, 63 aa, Peptostreptococcus magnus
EEVTIKANLIYADGKTQTAEFKGTFEEATAEAYRYADLLAKENGKYTVDVADKGYTLNIKFAG

SEQ ID NO: 15 - protein, 63 aa, Peptostreptococcus magnus
EEVTIKANLIYADGKTQTAEFKGTFAEATAEAYRYADLLAKENGKYTADLEDGGYTINIRFAG

SEQ ID NO: 16 - protein, 63 aa, Peptostreptococcus magnus
EQVTIKENIYFEDGTVQTATFKGTFAEATAEAYRYADLLSKEHGKYTADLEDGGYTINIRFAG

SEQ ID NO: 17 - protein, 64 aa, Peptostreptococcus magnus
EEVTIKANLIFADGSTQNAEFKGTFAKAVSDAYAYADALKKDNGEYTVDVADKGLTLNIKFAGK

SEQ ID NO: 18 - protein, 63 aa, Peptostreptococcus magnus
EEVTIKVNLIFADGKTQTAEFKGTFEEATAKAYAYADLLAKENGEYTADLEDGGNTINIKFAG

SEQ ID NO: 19 - protein, 63 aa, Peptostreptococcus magnus
EEVTIKVNLIFADGKIQTAEFKGTFEEATAKAYAYANLLAKENGEYTADLEDGGNTINIKFAG

SEQ ID NO: 20 - protein, 64 aa, Peptostreptococcus magnus
EEVTIKVNLIFADGKTQTAEFKGTFEEATAEAYRYADLLAKVNGEYTADLEDGGYTINIKFAGK

SEQ ID NO: 21 - protein, 63 aa, Artificial Sequence
  engineered PpL analogue
  MISC_FEATURE (2)..(2): Xaa at position 2 is Gln or Glu;
  MISC_FEATURE (7)..(7): Xaa at position 7 is Glu, Ala or Val;
  MISC_FEATURE (9)..(9): Xaa at position 9 is Ile or Leu;
  MISC_FEATURE (10)..(10): Xaa at position 10 is Tyr or Ile;
  MISC_FEATURE (11)..(11): Xaa at position 11 is Phe or Tyr;
  MISC_FEATURE (12)..(12): Xaa at position 12 is Ala or Glu;
  MISC_FEATURE (13)..(13): Xaa at position 13 is Asp or Asn;
  MISC_FEATURE (15)..(15): Xaa at position 15 is Thr, Ser or Lys;
  MISC_FEATURE (16)..(16): Xaa at position 16 is Val,Ile or Thr;
  MISC_FEATURE (18)..(18): Xaa at position 18 is His, Thr or Asn;
  MISC_FEATURE (20)..(20): Xaa at position 20 is Thr or Glu;
  MISC_FEATURE (26)..(26): Xaa at position 26 is Ala or Glu;
  MISC_FEATURE (27)..(27): Xaa at position 27 is Glu or Lys;
  MISC_FEATURE (29)..(29): Xaa at position 29 is Thr or Val;
  MISC_FEATURE (30)..(30): Xaa at position 30 is Ala or Ser;
  MISC_FEATURE (31)..(31): Xaa at position 31 is Glu, Asp or Lys;
  MISC_FEATURE (34)..(34): Xaa at position 34 is Arg or Ala;
  MISC_FEATURE (37)..(37): Xaa at position 37 is Asp or Asn;
  MISC_FEATURE (38)..(38): Xaa at position 38 is Leu, Thr or Ala;
  MISC_FEATURE (40)..(40): Xaa at position 40 is Ser, Lys or Ala;
  MISC_FEATURE (42)..(42): Xaa at position 42 is Glu, Asp or Val;
  MISC_FEATURE (43)..(43): Xaa at position 43 is His or Asn;
  MISC_FEATURE (45)..(45): Xaa at position 45 is Lys or Glu;
  MISC_FEATURE (48)..(48): Xaa at position 48 is Ala or Val;
  MISC_FEATURE (50)..(50): Xaa at position 50 is Leu or Val;
  MISC_FEATURE (51)..(51): Xaa at position 51 is Glu or Ala;
  MISC_FEATURE (53)..(53): Xaa at position 53 is Gly or Lys;
  MISC_FEATURE (55)..(55): Xaa at position 55 is Tyr, Leu or Asn;
  MISC_FEATURE (57)..(57): Xaa at position 57 is Ile or Leu;
  MISC_FEATURE (60)..(60): Xaa at position 60 is Arg or Lys;
EXVTIKXNXXXXXGXXQXAXFKGTFXXAXXXAYXYAXXLXKXXGXYTXDXXDXGXTXNIXFAG

SEQ ID NO: 22 - protein, 301 aa, Artificial Sequence
  PpL Mutant
KEETPETPETDSEEQVTIKENIYFEDGTVQHATFKGTFAEATAEAYRYADLLSKEHGKYTADLEDGGYTINIRFAGKEKTPEEPKEQVTIKENIYFEDGTVQHATFKGTFAEATAEAYRYADLLSKEHGKYTADLEDGGYTINIRFAGKEKTPEEPKEQVTIKENIYFEDGTVQHATFKGTFAEATAEAYRYADLLSKEHGKYTADLEDGGYTINIRFAGKEKTPEEPKEQVTIKENIYFEDGTVQHATFKGTFAEATAEAYRYADLLSKEHGKYTADLEDGGYTINIRFAGKKVDEKPEE

SEQ ID NO: 23 - protein, 63 aa, Artificial Sequence
  PpL Mutant
EQVTIKENIYFEDGTVQHATFKGTFAEATAEAYRYADLLSKEHGKYTADLEDGGYTINIRFAG

SEQ ID NO: 24 - protein, 302 aa, Artificial Sequence
  PpL Mutant
KEETPETPETDSEEQVTIKENIYFEDGTVQHATFKGTFAEATAEAYRYADLLSKEHGKYTADLEDGGYTINIRFAGKEKTPEEPKEQVTIKENIYFEDGTVQHATFKGTFAEATAEAYRYADLLSKEHGKYTADLEDGGYTINIRFAGKEKTPEEPKEQVTIKENIYFEDGTVQHATFKGTFAEATAEAYRYADLLSKEHGKYTADLEDGGYTINIRFAGKEKTPEEPKEQVTIKENIYFEDGTVQHATFKGTFAEATAEAYRYADLLSKEHGKYTADLEDGGYTINIRFAGKKVDEKPEEC

SEQ ID NO: 25 - DNA, 906 bp, Artificial Sequence
  DNA code
aaagaagaaaccccggaaacacctgaaactgattccgaagaacaagtaaccattaaagagaacatctacttcgaagatggcacagtacagcatgccacgtttaagggaacctttgctgaggctaccgccgaggcataccgttatgcggacttactgtctaaggaacacggaaagtacaccgctgacttagaagatggagggtatactatcaatattcgcttcgccggtaaagaaaaaactccggaagaacccaaagaacaggtgactatcaaggaaaacatttattttgaggacggcaccgtgcaacatgcgacgtttaaaggcaccttcgccgaagcaaccgcagaagcatatcgctatgcggatctgctcagtaaggaacatgggaaatataccgccgatctggaagacggtggctacacaattaacatacgatttgcgggtaaggaaaaaaccccagaggagccaaaagagcaggttaccattaaggagaatatttatttcgaggatgggaccgtccagcatgctacgttcaaaggtacgtttgcggaggcgacggcagaggcctatcgctacgccgatctactgagcaaagaacacggtaaatacactgcggaccttgaggatggcggttacacgattaacattcgtttcgcgggcaaagagaaaacgcccgaagaaccgaaggaacaagtcactatcaaagaaaatatctactttgaagacggaacggttcagcacgctaccttcaagggtacctttgcagaagccacagcggaagcctatcggtatgcagacctgctgtcgaaagagcatggcaaatacacggcggatttggaagatgggggctataccatcaatatccgttttgctggtaaaaaagtggatgagaaaccggaagaatgc

SEQ ID NO: 26 - DNA, 923 bp, Artificial Sequence
  DNA code
ctgcaggaaaagaagaaaccccggaaacacctgaaactgattccgaagaacaagtaaccattaaagagaacatctacttcgaagatggcacagtacagcatgccacgtttaagggaacctttgctgaggctaccgccgaggcataccgttatgcggacttactgtctaaggaacacggaaagtacaccgctgacttagaagatggagggtatactatcaatattcgcttcgccggtaaagaaaaaactccggaagaacccaaagaacaggtgactatcaaggaaaacatttattttgaggacggcaccgtgcaacatgcgacgtttaaaggcaccttcgccgaagcaaccgcagaagcatatcgctatgcggatctgctcagtaaggaacatgggaaatataccgccgatctggaagacggtggctacacaattaacatacgatttgcgggtaaggaaaaaaccccagaggagccaaaagagcaggttaccattaaggagaatatttatttcgaggatgggaccgtccagcatgctacgttcaaaggtacgtttgcggaggcgacggcagaggcctatcgctacgccgatctactgagcaaagaacacggtaaatacactgcggaccttgaggatggcggttacacgattaacattcgtttcgcgggcaaagagaaaacgcccgaagaaccgaaggaacaagtcactatcaaagaaaatatctactttgaagacggaacggttcagcacgctaccttcaagggtacctttgcagaagccacagcggaagcctatcggtatgcagacctgctgtcgaaagagcatggcaaatacacggcggatttggaagatgggggctataccatcaatatccgttttgctggtaaaaaagtggatgagaaaccggaagaatgctaatctaga

SEQ ID NO: 27 - protein, 301 aa, Artificial Sequence
  PpL Mutant
KEETPETPETDSEEQVTIKENIYFEDGTVQTATFKGTFAEATAEAYRYADLLSKEHGKYTADLEDGGYTINIRFAGKEKTPEEPKEQVTIKENIYFEDGTVQTATFKGTFAEATAEAYRYADLLSKEHGKYTADLEDGGYTINIRFAGKEKTPEEPKEQVTIKENIYFEDGTVQTATFKGTFAEATAEAYRYADLLSKEHGKYTADLEDGGYTINIRFAGKEKTPEEPKEQVTIKENIYFEDGTVQTATFKGTFAEATAEAYRYADLLSKEHGKYTADLEDGGYTINIRFAGKKVDEKPEE